(19) United States
(12) Patent Application Publication
(10) Pub. No.: US 2005/0163828 A1
(43) Pub. Date: Jul. 28, 2005

(54) Tocopheryl polyethylene glycol succinate articles and process for preparing TPGS articles

(76) Inventors: Bobby Lynn Bernard, Rogersville, TN (US); Stephen Hong-Wei Wu, Chesterfield, MO (US)

Correspondence Address: Jonathan D. Wood;Eastman Chemical Company, P.O. Box 511, Kingsport, TN, 37662-5075, US

(21) Appl. No.: 10/974,285

(22) Filed: Oct. 27, 2004

Related U.S. Application Data
(60) Provisional application No. 60/514,746, filed on Oct. 27, 2003.

(51) Int. Cl. A61L 15/16

(52) U.S. Cl. 424/448

(57) ABSTRACT

The present invention relates to a TPGS article having a weight no greater than 1 gram and a tackiness no greater than about 1550 grams. In addition, the present invention relates to a process for producing said TPGS articles.

This application claims the benefit of U.S. Provisional Application Ser. No. 60/514,746, filed Oct. 27, 2003.

The present invention relates to tocopheryl polyethylene glycol succinate (TPGS) articles that have increased hardness, reduced tackiness and improved handling characteristics.

### BACKGROUND OF THE INVENTION

Tocopheryl polyethylene glycol succinate (TPGS) has been used in various pharmaceutical compositions. For example, U.S. Pat. No. 3,102,078, discloses the use of TPGS as a solubilizing agent for water-soluble preparations of normally water-insoluble, fat-soluble vitamins. At room temperature TPGS is a waxy low melting solid that is very difficult to handle (Eastman Chemical Company, “Pharmaceutical Ingredients Eastman Vitamin E TPGS NF—Properties and Applications” (PCI-102, November 2002)). Typically TPGS is sold in the form of solid block containers. Whenever TPGS is to be used, the entire container is heated to a temperature above the melting temperature of TPGS (37-41° C.) and poured out in the desired amount as a liquid. Although TPGS is heat-stable having a decomposition temperature of about 200° C., repeated heating and cooling cycles of the material can cause discoloration and change the composition resulting in a decreased shelf life for TPGS. In addition, it is inconvenient for the customer to melt the entire TPGS for each use, requiring added time and equipment.

Alternatively TPGS can be removed from the container by breaking the solid cake or by other mechanical means. However, this means of removing the TPGS is inconvenient and often results in contamination even under good manufacturing practices, which is particularly a concern in the pharmaceutical environment. Furthermore, it is hard to be quantitative in removing an amount of solid from a waxy solid block.

U.S. Pat. No. 5,179,122 describes a solid composition comprising vitamin E acetate, TPGS and an inert carrier, wherein the TPGS is absorbed or adsorbed onto or into a solid inert carrier such as microcrystalline cellulose, starch, and inorganic materials. This is useful when a mixture of TPGS and another component is acceptable, however, there remains a need for a homogeneous TPGS form that is easy to handle. The applications of TPGS with a carrier are certainly limited when the carrier is not a desirable component in a formulation.

In view of the above handling difficulties with TPGS it is desirable to have TPGS in a form which can be handled easily.

### BRIEF SUMMARY OF THE INVENTION

The present invention overcomes the above mentioned problems by providing a TPGS article having improved surface tackiness and increased hardness characteristics.

The present invention relates to an article consisting essentially of TPGS, wherein said article has a weight no greater than 1 gram, and a surface tackiness no greater than 1550 grams. In a preferred embodiment the article has a weight from about 10 mg to about 150 mg, a more preferred embodiment has a weight from about 15 mg to about 90 mg and most preferably has a weight between 20 mg and 80 mg. The surface tackiness is preferably no greater than 1500 grams, more preferably no greater than about 1485 grams and most preferably no greater than about 1450 grams.

Another embodiment of the present invention is a process for producing TPGS articles having a weight no greater than about 1 gram, and preferably a weight from about 10 mg to about 250 mg, comprising the steps of providing TPGS at a temperature above its melting point; and forming the TPGS article at a temperature below about 31 degrees Celsius (C). The TPGS article is preferably formed at a temperature below about 20 degrees C., more preferably at a temperature below about 5 degrees C., more preferably at a temperature below about 0 degrees C. or below, and most preferably at a temperature below about −4 degrees C.

### DETAILED DESCRIPTION

TPGS can be prepared by esterifying tocopheryl acid succinate (herein TAS) with polyethylene glycol (herein PEG). U.S. Pat. No. 2,680,749 (incorporated herein by reference), provides method for preparing TPGS. The esterification procedure is preferably performed in a solvent media; promoted by any well known esterification catalyst; and performed under conditions such that the water formed during the esterification will be removed by azeotropic distillation. The polyethylene glycol (PEG) used in the esterification of the tocopheryl acid succinate (TAS) preferably has a number average molecular weight ranging from about 300 to about 6000, with a preferred range of from about 400 to about 1500. The resulting product comprises at least polyethylene glycol esters of tocopheryl acid succinate and unreacted PEG. The esters comprise at least mono-ester tocopheryl polyethylene glycol succinate (mono-TPGS), which is the major component, and di-esters of tocopheryl polyethylene glycol succinate (di-TPGS). Thus a TPGS product contains at least these three components, mono-TPGS, di-TPGS and residual PEG.

When TPGS is in its liquid state, the composition of the mono-TPGS, di-TPGS and PEG are uniformly present. In addition it was found that in its solidified form (TPGS below its melting point) the composition of the mono-TPGS, di-TPGS and PEG are all uniformly present in the TPGS.

The TPGS articles can be in any form below the melting point of TPGS, such as flakes, pellets and the like. The size of the articles are such that they may be easily handled. For example, the articles should be small enough to be handled easily, such as pouring in desired quantities, yet large enough that they do not stick together. It has been found that very small fines have a tendency to stick together. In its broadest embodiment, the size of the articles is equal to or less than about 1 gram. In a preferred embodiment the TPGS is in a flake form having a weight from about 10 mg to about 150 mg, preferably from about 15 mg to about 90 mg, and most preferably from about 20 mg to about 80 mg.

In a preferred embodiment, TPGS articles, such as flakes can be made by the following steps:

a. Melting the solid to form a liquid. The melting can be performed by any means, such as by heating TPGS to a temperature above its melting temperature to form a liquid.

b. Forming liquid particles, such as droplets. The particles can be formed by allowing the liquid to flow through a nozzle or other device which forms liquid droplets.

c. Cooling the particles to form TPGS articles. The particles can be cooled by contacting the liquid droplets with a cooler environment such as cool air or inert gas, contacting the liquid droplets with an object such as the surface of a drum or conveying belt that is cooler than the particles and that will form a handleable particle. The temperature of the environment of surface temperature of about 31 degrees C., preferably below that will quench the TPGS. This can also be accomplished spraying the liquid from a tower containing a spray nozzle wherein liquid droplets enters an inert atmosphere or atmosphere of air having a temperature of about 31 degrees or below that will quench the TPGS. The formation of the TPGS article described herein is not limited to the above described processes, but is applicable to any process whereby the TPGS article is formed.

For step a), the temperature is in the range from about 40 C to about 85° C., preferably from about 45 C to about 75° C., and most preferably a range from about 45 C to about 55° C. The liquid TPGS has a viscosity in the range of about 100 cps to about 500 cps (centi-poise/sec) and a preferred viscosity range from about 200 cps to about 400 cps.

For step b), any conventional equipment for making a flake article can be used. For example, typical equipment consists of rotary drum flaker with inert atmosphere and associated temperature control devices for the drum, inert melting tank with heating source and control, and heat controlled inter-connecting line from melt tank to the flaker.

For step c), the atmosphere or object having a temperature of about 31 degrees C. or below can be any atmosphere or surface that will quench the TPGS, such as a metal surface of a drum, a conveying beltor a tower with spray nozzle can also be used. A cooling media can be used to maintain the object at a temperature of about 0 degrees C. or below, such as chilled water or a brine glycol system that can provide a temperature in the range of 25 to −24° C. Temperature and cooling media selection depends on particular application configuration and desired output. The droplet size in step b) and then becoming a discrete particle in step c) upon cooling can have any weight and shape easy to be handled.

The invention will be more readily understood by reference to the following examples. There are, of course, many other forms of this invention which will become obvious to one skilled in the art, once the invention has been fully disclosed, and it will accordingly be recognized that these examples are given for the purpose of illustration only, and are not to be construed as limiting the scope of this invention in any way.

### EXAMPLES

Methods of Analysis

The following test procedures were used in evaluating the analytical properties of the products herein.

Differential scanning calorimetry (DSC) was used for determining the melting temperature of TPGS. The instrument used was a Mettler differential scanning calorimeter (Model 821, Mettler Toledo Inc., Columbus, Ohio). A TPGS sample of about 4.8 mg was weighed and placed on a 40 micrometer pan and hermetically sealed. The heating and cooling cycles were set between −140° C. and 85° C. with a 20° C./min heating rate. Cooling was done by liquid nitrogen purge (30 ml/min.) at temperatures from about 15 degrees C. to about −130 degrees C., for 10 minutes isothermally at −130 degrees C., then heating to 75 degrees C., held for 1 minute and then cooled back down to −130 degrees C. and held isothermally for 10 minutes. A second cycle was then run from −130 degrees C. to 75 degrees C. All the cycles with a heating and cooling rate of 20 degrees C./minute. The melting temperature of TPGS was then determined by the temperature at which abrupt changes of heat absorption curve occurred.

The compositions of TPGS were determined by an HPLC method using the following typical conditions.

| | |
|---|---|
| Column: | Inertsil C8, 4.6 mm ID × 150 mm L, 5 μ particles |
| Flow rate: | 1.0 mL/min |
| Mobile phases: | A: 50/50 CH3CN/2-PrOH (v/v) |
| | B: 100% deionized H2O |
| Gradient program: | equil: 60% A |
| | step 1: 60% A to 100% A, 40 min, linear |
| | step 2: 100% A, hold for 10 min |
| | step 3: 100% A to 60% A, 1 min, linear |
| Detection: | UV, @ 280 nm |
| Injection volume: | 20 μL |

The viscosity is measured by a Brookfield viscometer with heat control. The surface hardness and surface tackiness (also referred to as dragging tension) were measured by using a texture analyzer (Model, TA-XT2, Texture Technologies Corp., Scarsdale, N.Y.).

The tackiness test, utilized a “SMS Chen-Hoseney Dough Stickiness Rig” on the TA-XT2 Texture Analyzer. The method applies a fixed pressure on a 25 mm cylinder probe in a specified weight of sample against a solid metal mass. After applying the fixed pressure on the sample for a specific time, the amount of energy needed to withdraw the cylinder is measured (grams). In this particular application 2000 grams of pressure was exerted on the probe against the sample for 60 seconds.

The breaking test utilized a “Three Point Bend Ring” on the TA-XT2 Texture Analyzer. A base with two protruding arms mounts to the analyzer. A bar is attached to the arm applying force. A sample is placed on the arms mounted to the base and the force necessary to break the sample is recorded on a graph. In this application advancement of the arm applying the force was at a rate of 0.5 mm per second.

### Example 1

There was provided a vitamin E TPGS NF d-alpha-tocopheryl polyethylene glycol succinate, commercially available from Eastman Chemical Company (TPGS). The average composition, analyzed by a HPLC method, comprises:

| | |
|---|---|
| Mono-TPGS | approximately 80.0 wt % |
| Di-TPGS | approximately 10.0 wt % |
| Residual PEG | approximately 5.0 wt % |
| Vitamin E | approximately 0.2 wt % |
| Others | approximately 5 wt % |

The TPGS product exhibited an average molecular weight (MWn) of 1,513 and a MP of 38.0° C. measured by DSC.

### Example 2

TPGS from Example 1 was used to form TPGS article samples prepared at three temperatures, −24, −4 and 24° C. respectively, for analysis by the following method. A 1-kilogram container containing TPGS in its waxy solid form was heated at 55° C. in a convection oven. The liquid TPGS was then applied dropwise to glass plates that were pre-cooled at the above listed three temperatures (42 degrees C., −4 degrees C. and −24 degrees C.).

The range of the droplet weight was approximately 20-80 mg. The droplets immediately solidified and formed a flat flake shape articles on the plates pre-cooled to −24 and −4° C. The droplets also formed a flake particle on the plate treated at 24° C., but at a slower rate.

After applying TPGS droplets onto the plates, the plates were covered and held for one hour to equilibrate at room temperature. The TPGS flakes on the plates were then removed by using a razor knife. The majority of the articles were in flake form with some fine particles. Each article exhibited a weight in the range of about 20 to 80 mg. The fine particles have a weight of less than 20 mg for each particle.

### Example 3

The compositions of TPGS articles and the starting materials were analyzed by a HPLC analytical method as described at the beginning of the Examples. The results are shown in the following tables. Table 1 shows the compositions of the TPGS articles produced at the three temperatures with a sample of TPGS removed from the 1-Kg container before it was melted and processed in Example 2. Table 2 shows the compositions of TPGS samples taken from the selected locations in a 1-Kg container.

TABLE 1
Compositions of Flaked TPGS and Bulk TPGS sample in the Container

| | | Di-TPGS | Residual PEG |
|---|---|---|---|
| Sample | Mono-TPGS wt % | wt % | wt % |
| Original TPGS Cake | 81.32 | 11.19 | 5.39 |
| −24° C. | 81.19 | 11.28 | 5.36 |
| −4° C. | 81.44 | 11.32 | 5.50 |
| 24° C. | 81.29 | 11.31 | 5.25 |

A TPGS “cake” was prepared in the cake container having the dimensions of 7.5 inch in diameter and 1.5 inch in depth. TPGS was melted at about 55 degrees C. and poured into the cake container and allowed to cool to form a waxy solid having the dimensions of approximately equivalent to that of the cake container. Five samples were taken from the cake at the following locations:

A: Sample at top surface. Sample A was obtained by shaving the top surface area to a depth of 1/16 of an inch with a razor knife.

B: Sample at the bottom surface. Sample B was obtained by shaving the bottom surface area to a depth of 1/16 of an inch with a razor knife.

C: Sample at middle section close to the side of the container.

D: Sample at the upper area below the top surface.

E: Sample at the middle section of the container.

TABLE 2
Compositions of TPGS Samples at Selected Locations
in a 1-Kg Container

| Sample | Mono-TPGS wt % | Di-TPGS % | Residual PEG wt % |
|---|---|---|---|
| A | 81.42 | 11.33 | 5.54 |
| B | 81.11 | 11.31 | 5.43 |
| C | 81.29 | 11.31 | 5.32 |
| D | 81.34 | 11.33 | 5.38 |
| E | 81.43 | 11.38 | 5.39 |

### Example 4

This example shows the hardness of TPGS articles solidified at the specified temperatures in Example 2 using the Breaking Test described above.

Three TPGS samples each weighting 90 grams and measuring 80 mm×80 mm×16 mm thickness, were produced by melting the TPGS at 50° C., and then pouring the entire liquefied sample (90 grams) into the pans of the same dimensions, and then cooling the samples immediately at the specified temperatures (24 degrees C., −4 degrees C., and −24 degrees C.). The samples were held at each temperature for 12 hours, and then equilibrated to 24° C. over a period of 24 hrs.

The Breaking Test was performed using a TA-XT2 Texture Analyzer. The hardness was measured by the amount of force, measured in Newton, needed to break the article. The results are shown in Table 3.

TABLE 3
Hardness of TPGS Samples in Cake Form Prepared at Three Selected
Temperatures

| TPGS | Solidifying | Breaking Force, |
|---|---|---|
| Sample | Temperature, ° C. | Newton |
| A | −24 | 242.23 |
| B | −4 | 235.14 |
| C | 24 | 176.25 |

### Example 5

This example shows the surface tackiness of TPGS articles using the tackiness test described above, wherein the articles were solidified at the specified temperatures (24 degrees C., −4 degrees C. and −24 degrees C.).

Three TPGS article samples were prepared according to Example 2 were analyzed. The measurement of surface tackiness was performed on a TA-XT2 Texture Analyzer using a 1 inch cylinder probe. A small amount of the TPGS article was placed on a flat metal surface and the sample was compressed by the cylinder for 60 second. The compression force was 2 Kg. Then the cylinder was withdrawn at 0.1 mm/sec. The surface tackiness (dragging tension) was measured upon withdrawal of the probe. The dragging tension, measured in grams, is a measure of surface tackiness. The results are shown in Table 4.

TABLE 4
Surface Tackiness of TPGS Flake Samples Prepared at Three Selected
Temperatures

| TPGS | Solidifying | Dragging Tension, |
|---|---|---|
| Sample | Temperature, ° C. | grams |
| A | −24 | 1443.9 |
| B | −4 | 1485.3 |
| C | 24 | 1620.4 |

### Example 6

The melting temperatures (Tm) and crystallization temperatures (Tc) of Sample A, B and C from Example 4 were measured by a DSC method described at the beginning of Examples session. The results are shown in the following Table 5.

TABLE 5
Tm and Tc of TPGS Samples in Cake Form Prepared at Three Solidifying
Temperatures

| | | | |
|---|---|---|---|
| | | | Crystallization |
| TPGS | Solidifying | Melting Temperature | Temperature |
| Sample | Temperature, ° C. | (Tm), ° C. | (Tc), ° C. |

| | | | |
|---|---|---|---|
| A | −24 | 36.2 | 6.45 |
| B | −4 | 37.1 | 9.51 |
| C | 24 | 38.0 | 13.0 |

1. An article consisting essentially of TPGS, wherein said article has a weight no greater than about 1 gram, and a surface tackiness no greater than about 1550 grams.

2. An article as recited in claim 1, wherein the surface tackiness in no greater than about 1485.

3. An article as recited in claim 1, wherein the surface tackiness in no greater than about 1450.

4. An article as recited in claim 1, wherein the article has a weight from about 10 mg to about 150 mg.

5. An article as recited in claim 1, wherein the article has a weight from about 15 mg to about 90 mg.

6. An article as recited in claim 1, wherein the article has a weight from about 20 mg to about 80 mg.

7. A process for producing TPGS articles having a weight no greater than about 1 gram, comprising the steps of:
providing TPGS at a temperature above its melting point; and
forming the TPGS article at a temperature below about 31 degrees Celsius (C).

8. A process as recited in claim 7, wherein the step of forming the TPGS article is at a temperature below about 20 degrees C.

9. A process as recited in claim 7, wherein the step of forming the TPGS article is at a temperature below about 5 degrees C.

10. A process as recited in claim 7, wherein the step of forming the TPGS article is at a temperature below about 0 degrees C.

11. A process as recited in claim 7, wherein the step of forming the TPGS article is at a temperature below about 4 degrees C.

12. A process as recited in claim 7, wherein the weight of the TPGS article is from about 10 mg to about 25 mg.
